(19) United States
(12) Patent Application Publication
(10) Pub. No.: US 2020/0211706 A1
(43) Pub. Date: Jul. 2, 2020

(54) INTELLIGENT TRADITIONAL CHINESE MEDICINE DIAGNOSIS METHOD, SYSTEM AND TRADITIONAL CHINESE MEDICINE SYSTEM

(71) Applicant: GUANGDONG UNIVERSITY OF TECHNOLOGY, Guangzhou, Guangdong (CN)

(72) Inventors: Everett Xiaolin WANG, Guangzhou, Guangdong (CN); Anhui LIANG, Guangzhou, Guangdong (CN); Xiaosa WANG, Guangzhou, Guangdong (CN); Hongwu WANG, Guangzhou, Guangdong (CN); Bin CAO, Guangzhou, Guangdong (CN); Shiyu LI, Guangzhou, Guangdong (CN); Yonghuang WU, Guangzhou, Guangdong (CN); Mei LIU, Guangzhou, Guangdong (CN); Dong CAO, Guangzhou, Guangdong (CN)

(73) Assignee: GUANGDONG UNIVERSITY OF TECHNOLOGY, Guangzhou, Guangdong (CN)

(21) Appl. No.: 16/634,135

(22) PCT Filed: Dec. 14, 2017
(86) PCT No.: PCT/CN2017/116065

§ 371 Date: Jan. 25, 2020

(30) Foreign Application Priority Data
Jul. 31, 2017 (CN) 201710639260.7

(51) Int. Cl. G16H 50/20; G16H 20/90; G16H 10/60; G16H 50/70; G16H 70/60; G16H 70/20; G16H 50/50; G16H 20/30; G06N 3/08; G06N 3/04

(52) U.S. Cl. CPC G16H 50/20; G16H 20/90; G16H 10/60; G16H 50/70; G16H 70/60; G06N 3/0472; G16H 50/50; G16H 20/30; G06N 3/08; G06N 3/0454; G16H 70/20

(57) ABSTRACT

Disclosed are an intelligent traditional Chinese medicine diagnosis method, system and traditional Chinese medicine system, the method comprising: a server side obtaining, from distributed client clusters, inspection training data, auscultation-olfaction diagnosis training data, interrogation training data and palpation training data of a patient; the server side training, by using the inspection training data, the auscultation-olfaction diagnosis training data, the interrogation training data and the palpation training data, a model to be trained established on the basis of a deep neural network algorithm to obtain a trained model; and the server side using the trained model to perform diagnosis with respect to disease data of the patient to obtain a diagnostic result of the disease data. The technical solution disclosed by the present application can be used to comprehensively obtain disease information of a patient, thereby effectively increasing accuracy of the medical diagnostic result; in addition, the method can be used to quickly process disease data of multiple patients at the same time.

The present application claims the priority to Chinese Patent Application No. 201710639260.7, titled “INTELLIGENT TRADITIONAL CHINESE MEDICINE DIAGNOSIS METHOD, SYSTEM AND TRADITIONAL CHINESE MEDICINE SYSTEM”, filed on Jul. 31, 2017, with the China National Intellectual Property Administration, which is incorporated herein by reference in its entirety.

### TECHNICAL FIELD

The present disclosure relates to the technical field of disease diagnosis, and in particular to an intelligent traditional Chinese medicine diagnosis method, an intelligent traditional Chinese medicine diagnosis system, and a traditional Chinese medicine medical system.

### BACKGROUND

Traditional Chinese medicine treatment in China is one of the most distinctive methods of treating diseases in clinical medicine. A traditional Chinese medicine physician can obtain various disease information about human diseases by performing diagnosis through inspection, auscultation-olfaction, inquiry, and palpation on a patient, and then determine a treatment plan for the patient by comprehensively analyzing the obtained various disease information. Compared with western medicine treatment, traditional Chinese medicine treatment is safer and has more stable effects. In recent years, with the continuous development of modern medical technology, people are increasingly hoping to combine the traditional Chinese medicine treatment method with artificial intelligence, thereby reducing workload of the traditional Chinese medicine physician. At present, many medical systems have been developed to assist with diagnosis of traditional Chinese medicine. However, in current common medical diagnosis systems, a diagnosis plan for the patient is often determined based on disease information about one aspect of the disease of the patient. Therefore, a diagnosis result often has low accuracy, and the disease may even be misdiagnosed, which arises a problem required to be solved urgently in the field of intelligent traditional Chinese medicine diagnosis systems.

### SUMMARY

In view of this, an objective of the present disclosure is to provide an intelligent traditional Chinese medicine diagnosis method and an intelligent traditional Chinese medicine diagnosis system, to improve the accuracy of intelligent traditional Chinese medicine diagnosis system. Specific solutions thereof are provided as follows.

An intelligent traditional Chinese medicine diagnosis method includes:

obtaining, by a sever, inspection diagnosis training data, auscultation-olfaction diagnosis training data, inquiry diagnosis training data, and palpation diagnosis training data of a patient from a distributed client cluster;

training, by the sever, a to-be-trained model, established based on a deep neural network algorithm, with the inspection diagnosis training data, the auscultation-olfaction diagnosis training data, the inquiry diagnosis training data and the palpation diagnosis training data, to obtain a trained model; and

diagnosing, by the server, disease data of the patient with the trained model to obtain a diagnosis result of the disease data.

Preferably, the method is implemented in a distributed client-server architecture or a cloud computing architecture.

Preferably, the training a to-be-trained model, established based on a deep neural network algorithm, with the inspection diagnosis training data, the auscultation-olfaction diagnosis training data, the inquiry diagnosis training data and the palpation diagnosis training data, to obtain a trained model, includes:

training a to-be-trained inspection diagnosis model, established based on a convolution neural network algorithm, with the inspection diagnosis training data, to obtain a trained inspection diagnosis model;

training a to-be-trained auscultation-olfaction diagnosis model, established based on a BP neural network algorithm, with the auscultation-olfaction diagnosis training data, to obtain a trained auscultation-olfaction diagnosis model;

training a to-be-trained inquiry diagnosis model, established based on a BP neural network algorithm, with the inquiry diagnosis training data, to obtain a trained inquiry diagnosis model; and

training a to-be-trained palpation diagnosis model, established based on a deep neural network algorithm, with the palpation diagnosis training data, to obtain a trained palpation diagnosis model.

Preferably, the training a to-be-trained model, established based on a deep neural network algorithm, with the inspection diagnosis training data, the auscultation-olfaction diagnosis training data, the inquiry diagnosis training data and the palpation diagnosis training data, to obtain a trained model, includes:

training a to-be-trained inspection diagnosis model, established based on a convolution neural network algorithm, with the inspection diagnosis training data, to obtain a trained inspection model;

training a to-be-trained auscultation-olfaction diagnosis model, established based on a BP neural network algorithm, with the auscultation-olfaction diagnosis training data, to obtain a trained auscultation-olfaction diagnosis model;

training a to-be-trained inquiry diagnosis model, established based on a BP neural network algorithm, with the inquiry diagnosis training data, to obtain a trained inquiry diagnosis model;

training a to-be-trained palpation diagnosis model, established based on a deep neural network algorithm, with the palpation diagnosis training data, to obtain a trained palpation diagnosis model; and

training a to-be-trained model, established based on a probabilistic neural network algorithm, with data outputted from output terminals of the trained inspection diagnosis model, the trained auscultation-olfaction diagnosis model, the trained inquiry diagnosis model, and the trained palpation diagnosis model, to obtain a trained model.

Preferably, the method further includes:

performing denoising and/or smoothing processing on the inspection diagnosis training data.

Preferably, the method further includes:

performing filtering and/or framing processing on the auscultation-olfaction diagnosis training data.

Preferably, before the diagnosing disease data of the patient with the trained model to obtain a diagnosis result of the disease data, the method further includes:

optimizing the trained model with new training data to improve accuracy of the trained model, where the new training data is disease data obtained after the diagnosis result of the patient is verified.

An intelligent traditional Chinese medicine diagnosis system is further provided according to the present disclosure, which includes:

a data obtaining module, configured for a server to obtain inspection diagnosis training data, auscultation-olfaction diagnosis training data, inquiry diagnosis training data, and palpation diagnosis training data of a patient from a distributed client cluster;

a model establishing module, configured for the server to train a to-be-trained model, established based on a deep neural network algorithm, with the inspection diagnosis training data, the auscultation-olfaction diagnosis training data, the inquiry diagnosis training data, and the palpation diagnosis training data, to obtain a trained model; and

a diagnosis result obtaining module, configured for the server to diagnose disease data of the patient with the trained model to obtain a diagnosis result of the disease data.

Further, a traditional Chinese medicine medical system is further provided according to the present disclosure, including the foregoing intelligent traditional Chinese medicine diagnosis system. The traditional Chinese medicine medical system further includes:

an intelligent traditional Chinese medicine treatment system, configured to determine a corresponding treatment plan based on the diagnosis result obtained by the intelligent traditional Chinese medicine diagnosis system, where

the intelligent traditional Chinese medicine treatment system is a treatment system trained by using a deep neural network algorithm, and a corresponding training sample includes a history diagnosis result and a corresponding treatment plan.

Preferably, the treatment plan determined by the intelligent traditional Chinese medicine treatment system includes a prescription of Chinese traditional patent medicine and/or a physical therapy plan.

Preferably, the deep neural network algorithm used for training the intelligent traditional Chinese medicine treatment system includes a convolution neural network algorithm.

In the present disclosure, first, the inspection diagnosis training data, the auscultation-olfaction diagnosis training data, the inquiry diagnosis training data, and the palpation diagnosis training data of the patient are obtained by the sever from the distributed client cluster. Then, the to-be-trained model, established based on the deep neural network algorithm, is trained by the sever with the inspection diagnosis training data, the auscultation-olfaction diagnosis training data, the inquiry diagnosis training data, and the palpation diagnosis training data to obtain the trained model. Finally, the disease data of the patient is diagnosed by the server with the trained model to obtain a diagnosis result of the disease data. In the present disclosure, data to be inputted into an input terminal of the to-be-trained model is the inspection diagnosis training data, the auscultation-olfaction diagnosis training data, the inquiry diagnosis training data, and the palpation diagnosis training data of the patient collected from the distributed client cluster. Apparently, in this way, a large amount of disease data of the patient is obtained, and as the disease data is obtained by different means, the disease data is interrelated and restrained by each other, which can more comprehensively reflect a disease status of the patient. Compared with inputting one type of disease data into the input terminal of the to-be-trained model, the model inputted with the large amount of disease data has higher training accuracy. Moreover, the method according to the present disclosure is applied in the distributed client-server architecture. Thus, the training accuracy of the model is improved since more disease data of the patient can be obtained, and a faster diagnosis speed can be achieved when diagnosing the disease data of the patient with the model.

### BRIEF DESCRIPTION OF THE DRAWINGS

In order to describe the technical solutions in the embodiments of the present disclosure or in the conventional art more clearly, drawings to be used in the description of the embodiments or the conventional art are described briefly. Apparently, the drawings described below only show some of embodiments of the present disclosure, and for those skilled in the field, other drawings may be obtained from these drawings without any creative effort.

FIG. 1 is a flow chart of an intelligent traditional Chinese medicine diagnosis method according to a first embodiment of the present disclosure;

FIG. 2 is a flow chart of an intelligent traditional Chinese medicine diagnosis method according to a second embodiment of the present disclosure;

FIG. 3 is a flow chart of training a to-be-trained inspection diagnosis model;

FIG. 4 is a schematic structural diagram of a to-be-trained inspection diagnosis model;

FIG. 5 is a basic schematic structural diagram of an entire to-be-trained inspection diagnosis model;

FIG. 6 is a flow chart of preprocessing auscultation-olfaction diagnosis training data;

FIG. 7 is a schematic structural diagram of a to-be-trained auscultation-olfaction diagnosis model;

FIG. 8 is a basic schematic structural diagram of an entire to-be-trained auscultation-olfaction diagnosis model;

FIG. 9 is a basic schematic structural diagram of a to-be-trained inquiry diagnosis model;

FIG. 10 is a basic schematic structural diagram of an entire to-be-trained inquiry diagnosis model;

FIG. 11 is a flow chart of an intelligent traditional Chinese medicine diagnosis method according to a third embodiment of the present disclosure;

FIG. 12 is a basic schematic structural diagram of a probabilistic neural network;

FIG. 13 is a schematic structural diagram of an entire deep neural network algorithm;

FIG. 14 is a schematic diagram of a terminal cloud server; and

FIG. 15 is a schematic structural diagram of an intelligent traditional Chinese medicine diagnosis system according to an embodiment of the present disclosure.

### DETAILED DESCRIPTION OF THE EMBODIMENTS

The technical solutions according to the embodiments of the present disclosure are described clearly and completely hereinafter with reference to the drawings in embodiments of the present disclosure. Apparently, the described embodiments are only some rather than all of the embodiments of the present disclosure. Based on the embodiments of the present disclosure, all other embodiments obtained by those skilled in the art without any creative effort should fall within the protection scope of the present disclosure.

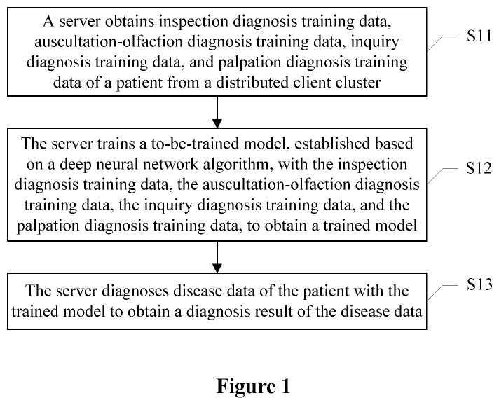

An intelligent traditional Chinese medicine diagnosis method is provided according to a first embodiment of the present disclosure. As shown in FIG. 1, the method includes the following steps S11 to S13.

In step S11, inspection diagnosis training data, auscultation-olfaction diagnosis training data, inquiry diagnosis training data, and palpation diagnosis training data of a patient are obtained by a sever from a distributed client cluster.

In the embodiment, the inspection diagnosis training data, the auscultation-olfaction diagnosis training data, the inquiry diagnosis training data, and the palpation diagnosis training data of the patient are obtained from the distributed client cluster located at multiple hospitals and clinics. In this way, all information about disease of the patient can be obtained more comprehensively, and then the obtained information about the disease of the patient is comprehensively analyzed to obtain a diagnosis result of the disease of the patient more accurately.

Moreover, the embodiment of the present disclosure is applied in a distributed client-server architecture. Thus, the training accuracy of the model is improved since more disease data of the patient can be obtained, and a faster diagnosis speed can be achieved when diagnosing the disease data of the patient. Furthermore, multiple patients can be diagnosed simultaneously by using the model, improving the practical performance of the model compared to the conventional art.

Further, preprocessing may be performed on the inspection diagnosis training data, the auscultation-olfaction diagnosis training data, and the palpation diagnosis training data to obtain more preferable training data to facilitate subsequent processing.

In step S12, a to-be-trained model established based on a deep neural network algorithm is trained by the sever with the inspection diagnosis training data, the auscultation-olfaction diagnosis training data, the inquiry diagnosis training data, and the palpation diagnosis training data, to obtain a trained model.

In the embodiment, the to-be-trained model is established based on a deep neural network algorithm. The deep neural network algorithm includes, but is not limited to, a convolution neural network algorithm, a BP neural network algorithm, and a probabilistic neural network algorithm that are common in the art. Besides, new neural network layers may be established at an input terminal and an output terminal of the deep neural network to optimize the trained model as established.

It should be understood that one neural network algorithm or several neural network algorithms may be adopted in the to-be-trained model as established, or several small neural network subsystems may be adopted in a large neural network system, as may be appropriate for purpose of solving practical problems.

It should be understood that the diagnosis result obtained with the method according to the embodiment of the present disclosure is more accurate than a diagnosis result obtained by processing only tongue image information of the patient with a convolution neural network. It should be understood that the tongue image information of the patient can only reflect a part of disease information of the patient, which results in less training data to be inputted into an input terminal of the model as established and thereby an inaccurate diagnosis result of the disease.

Moreover, compared with a conventional method of determining a treatment plan for the patient with an expert system, the diagnosis result will be more accurate with the method according to the embodiment of the present disclosure. It should be understood that the deep neural network algorithm has an ability of learning and relearning and can learn and generalize-summarize from known data. Therefore, it can be ensured that existing training data can be used with higher efficiency in training the mode. By contrast, such an effect cannot be achieved in determining the treatment plan with the expert system, since training data established even by an experienced expert is still limited, and a database established by the expert cannot include all diagnosis results corresponding to the disease information of the patient. Therefore, in comparison, the diagnosis result of the disease of the patient will be more accurate with the method.

In step S13, disease data of the patient is diagnosed by the server with the trained model to obtain a diagnosis result of the disease data.

In the embodiment, in the model established based on the deep neural network algorithm, the training data includes disease information obtained by performing four types of diagnosis on the patient: inspection, auscultation-olfaction, inquiry, and palpation. It should be understood that the disease data of the patient may be disease information about one aspect of the disease of the patient or disease information about several aspects of the disease of the patient. In this case, a corresponding diagnosis result will be obtained by the model established based on the deep neural network algorithm with the disease information provided by the patient. It should be understood that the diagnosis result in the embodiment is substantively a classification result obtained by an information processing device such as a computer based on deep learning, and is different from a diagnosis conclusion obtained by a physician based on medical theories.

Further, the trained model as established may be optimized with disease data corresponding to a verified diagnosis result of the patient, so that trained model can obtain more accurate diagnosis results and better diagnose diseases of patients. It should be noted that the disease data corresponding to the verified diagnosis result of the patient may be disease data corresponding to rehabilitation of the patient contained in a cloud server, or disease data corresponding to rehabilitation of the patient obtained by other means, where to-be-trained data for optimizing the trained model is not limited herein.

Moreover, the embodiment of the present disclosure is applied to a distributed client-server architecture. It is understood that the distributed client-server architecture can alleviate the problems of resource insufficiency and response bottlenecks at the client, and solve the problem of slow data operation speed in a centralized system.

Further, the intelligent traditional Chinese medicine diagnosis method according the embodiment may be implemented in a distributed client-server architecture or a cloud computing architecture.

In the present disclosure, first, the inspection diagnosis training data, the auscultation-olfaction diagnosis training data, the inquiry diagnosis training data, and the palpation diagnosis training data of the patient are obtained by the sever from the distributed client cluster. Then, the to-be-trained model, established based on the deep neural network algorithm, is trained by the sever with the inspection diagnosis training data, the auscultation-olfaction diagnosis training data, the inquiry diagnosis training data, and the palpation diagnosis training data to obtain the trained model. Finally, the disease data of the patient is diagnosed by the server with the trained model to obtain a diagnosis result of the disease data. In the present disclosure, data to be inputted into an input terminal of the to-be-trained model is the inspection diagnosis training data, the auscultation-olfaction diagnosis training data, the inquiry diagnosis training data, and the palpation diagnosis training data of the patient collected from the distributed client cluster. Apparently, in this way, a large amount of disease data of the patient is obtained, and as these disease data is obtained by different means, these disease data is interrelated and restrained by each other, which can more comprehensively reflect a disease status of the patient. Compared with inputting one type of disease data into the input terminal of the to-be-trained model, the model inputted with the large amount of disease data has higher training accuracy. Moreover, the method according to the present disclosure is applied in the distributed client-server architecture. Thus, the training accuracy of the model is improved since more disease data of the patient can be obtained, and a faster diagnosis speed can be achieved when diagnosing the disease data of the patient with the model.

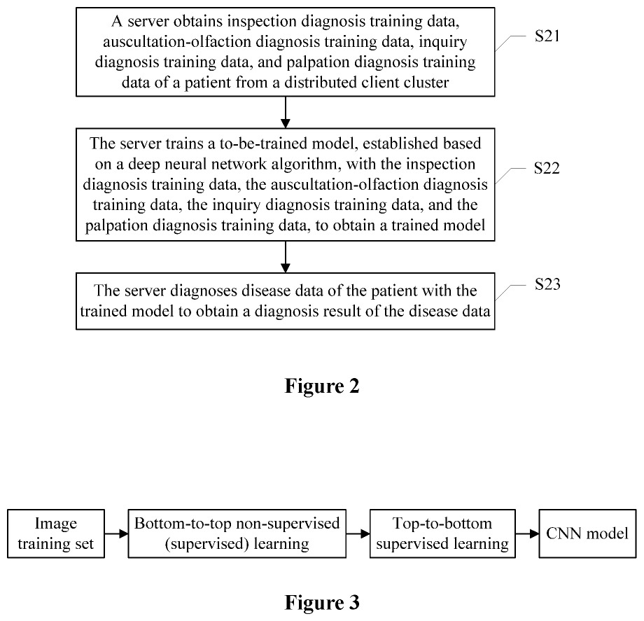

A specific intelligent traditional Chinese medicine diagnosis method is provided according to a second embodiment of the present disclosure. As shown in FIG. 2, compared with the foregoing embodiment, the technical solution is further explained and optimized in this embodiment, which includes the following steps S21 to S23.

In step S21, inspection diagnosis training data, auscultation-olfaction diagnosis training data, inquiry diagnosis training data, and palpation diagnosis training data of a patient are obtained by a sever from a distributed client cluster.

In the embodiment, the inspection diagnosis training data of the patient includes, but is not limited to, information about a face image and a tongue image of the patient. The auscultation-olfaction diagnosis training data of the patient includes, but is not limited to, talking voices, coughing, and wheezing of the patient. The inquiry diagnosis training data of the patient includes, but is not limited to, a disease cause and a medical history of the patient. The palpation diagnosis training data of the patient includes, but is not limited to, pulse information of the patient.

Disease data of the patient is obtained from a distributed client cluster, that is, the disease data of the patient is obtained from hospitals and clinics across the country. Thus, disease data samples for the model are more comprehensive. Then, a disease diagnosis result of the patient can be obtained more accurately by comprehensively analyzing all obtained disease information.

In step S22, a to-be-trained model established based on a deep neural network algorithm is trained by the sever with the inspection diagnosis training data, the auscultation-olfaction diagnosis training data, the inquiry diagnosis training data, and the palpation diagnosis training data, to obtain a trained model. In the embodiment, this step includes the following steps S221, S222, S223, and S224.

In step S221, a to-be-trained inspection diagnosis model established based on a convolution neural network algorithm is trained with the inspection diagnosis training data to obtain a trained inspection diagnosis model.

In the embodiment, a supervised learning mode is adopted in establishing the to-be-trained inspection diagnosis model based on a convolution neural network algorithm (Convolution Neural Network Algorithm, CNN). It should be understood that the inspection diagnosis training data is labeled data. A process of training the to-be-trained inspection diagnosis model is shown in FIG. 3.

Specifically, in the embodiment, a bottom-to-top non-supervised (supervised) learning mode is adopted for the to-be-trained inspection diagnosis model. First, parameters of layers of the to-be-trained inspection diagnosis model are trained hierarchically with uncalibrated data (or calibrated data). Then, the first layer of the to-be-trained inspection diagnosis model is trained with uncalibrated data (or calibrated data). It should be understood that when training the to-be-trained inspection diagnosis model, parameters of the first layer of the to-be-trained inspection diagnosis model are learned first. Then parameters of an (n−1)^{th} layer of the to-be-trained inspection diagnosis model are obtained by learning, and an output of the (n−1)^{th} layer of the to-be-trained inspection diagnosis model is used as an input of an n^{th} layer of the to-be-trained inspection diagnosis model to train the n^{th} layer of the to-be-trained inspection diagnosis model. Thus, the parameters of each layer of the to-be-trained inspection diagnosis model are obtained.

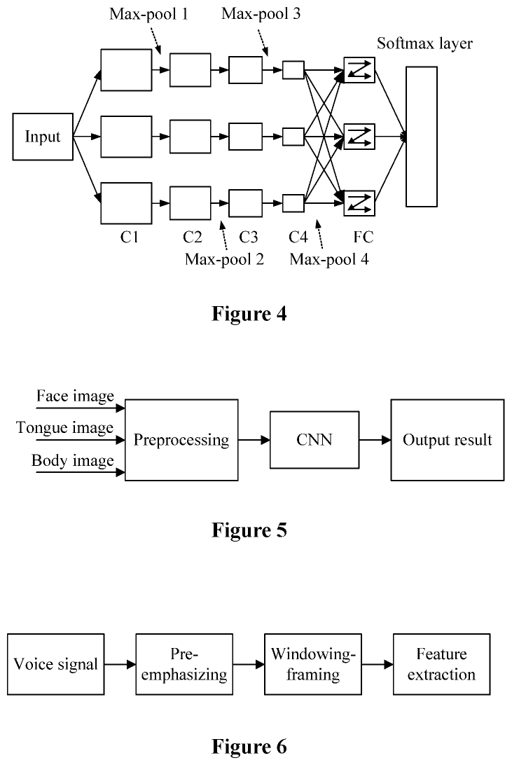

In a top-to-bottom supervised learning mode, the to-be-trained inspection diagnosis model is trained with labeled data first, an error is passed from top to bottom, and the neural network can be fine-tuned, so as to obtain a training and learning result of the to-be-trained inspection diagnosis model. An overall architecture of the to-be-trained inspection diagnosis model is shown in FIG. 4.

As shown in FIG. 4, C1, C2, C3, and C4 are convolutional layers of the to-be-trained inspection diagnosis model, where layer C1 has 96 11*11 convolution kernels, layer C2 has 256 5*5 convolution kernels, layer C3 has 384 3*3 convolution kernels, and layer C4 has 256 3*3 convolution kernels. The number of max-pooling layers in the to-be-trained inspection diagnosis model is 4, and convolution kernel of each max-pooling layer is 2*2 convolution kernel, where an output of a fourth max-pooling layer is used as an input of a fully connected layer. The fully connected layer links the output of the fourth max-pooling layer into a one-dimensional vector, and an output of the fully connected layer is classified at a softmax layer.

The parameters may be specifically configured in such other way as may be sufficient to achieve the purpose of practical application, which is not limited herein.

It should be noted that in the embodiment, inspection diagnosis training data, that is, a face image, a tongue image, and a body image of the patient, is inputted into the to-be-trained inspection diagnosis model. The above-mentioned images are all 3-channel images, that is, RGB images.

Further, in this step, denoising and/or smoothing processing may further be performed on the inspection diagnosis training data.

It should be understood that by performing the above processing, the inspection diagnosis training data is more preferable and is easier to be processed and analyzed in subsequent steps. A basic structure of the entire to-be-trained inspection diagnosis model is shown in FIG. 5.

In step S222, a to-be-trained auscultation-olfaction diagnosis model established based on a BP neural network algorithm is trained with the auscultation-olfaction diagnosis training data to obtain a trained auscultation-olfaction diagnosis model.

Specifically, in the embodiment, the auscultation-olfaction diagnosis training data may be obtained by, but not limited to, collecting voice data of the patient, including talking voices, coughing, and wheezing of the patient.

Further, in this step, filtering and/or framing processing may be further performed on the auscultation-olfaction diagnosis training data.

It should be understood that by performing the above processing, the inspection diagnosis training data is rendered more preferable. Specifically, pre-emphasizing processing on the auscultation-olfaction diagnosis training data is mainly to perform filtering and framing processing on a collected voice signal, and windowing-framing processing is to segment collected voice data to make the voice signal continuous and at a certain overlap ratio, which facilitates processing and analysis in subsequent steps. Moreover, in the embodiment, feature extraction is performed on preprocessed data, and an extracted feature vector is used as input data of the to-be-trained auscultation-olfaction diagnosis model, where zeros are filled for shortfall thereof. A process of preprocessing the auscultation-olfaction diagnosis training data is shown in FIG. 6.

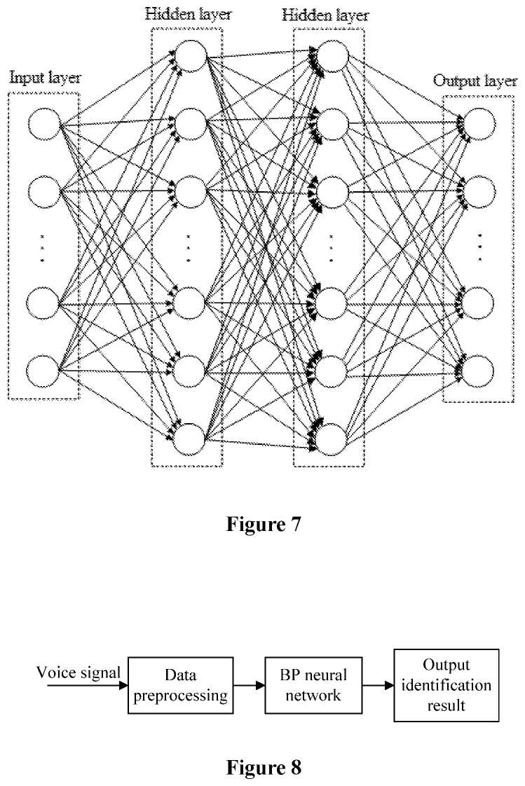

Reference is made to FIG. 7, which is a schematic structural diagram of a to-be-trained auscultation-olfaction diagnosis model. In the embodiment, the established to-be-trained auscultation-olfaction diagnosis model is a BP neural network having two hidden layers, where the number of input neurons is 600, the number of neurons in each output layer in the middle is 54, and the number of neurons in an output layer is 5. Learning process of the BP neural network includes two processes: forward transmission of signals and backward transmission of errors. In forward transmission, an input signal is inputted in the input layer and is transmitted to the output layer after being processed by each hidden layer. If an actual output of the output layer does not match an expected output (label), the process of backward transmission of errors is performed. In the process of backward transmission of errors, an output error is transmitted backward to the input layer via the hidden layers in a certain way and is distributed to all units of each layer, to obtain an error signal of each unit of each layer, where the error signal may be used as a basis for correcting a weight of each unit. By continuously correcting and adjusting a weight of each layer in the model in processes of forward transmission of signals and backward transmission of errors, training accuracy of the model can be continuously improved. A basic structure of the entire to-be-trained auscultation-olfaction diagnosis model is shown in FIG. 8.

In step S223, a to-be-trained inquiry diagnosis model established based on a BP neural network algorithm is trained with the inquiry diagnosis training data to obtain a trained inquiry diagnosis model.

In the embodiment, the inquiry diagnosis training data is obtained by the patient answering questions set by a system. For example, the questions set by the system include, but not limited to, age, gender, medical history, family, and living environment of the patient.

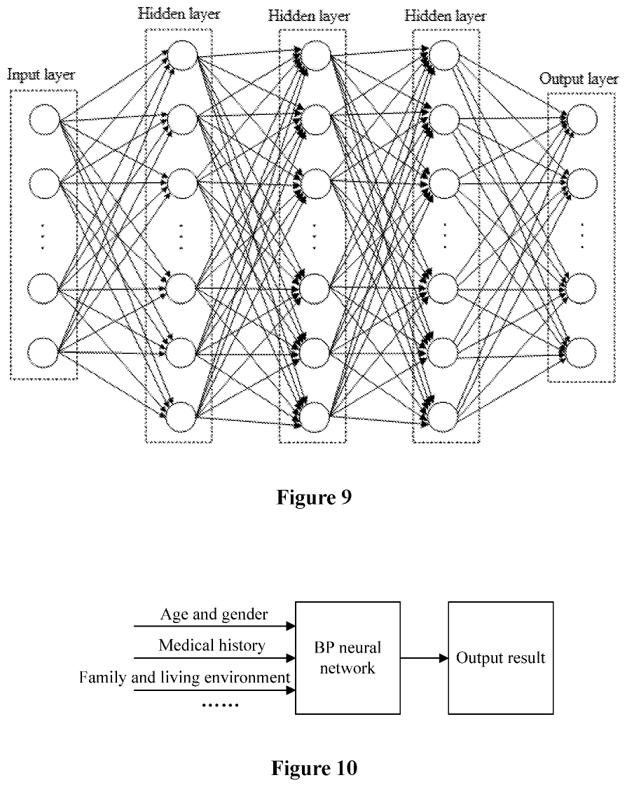

In the embodiment, the established to-be-trained inquiry diagnosis model is a BP neural network having three hidden layers, two input layers, and two output layers. A basic structure of the established to-be-trained inquiry diagnosis model is shown in FIG. 9. Specifically, the to-be-trained inquiry diagnosis model is a BP neural network having 8 inputs and 9 outputs, and the number of nodes in each hidden layer is set to 8. It should be understood that compared with setting a single hidden layer, setting multiple hidden layers can better ensure accuracy and stronger data generalization ability of the to-be-trained inquiry diagnosis model. A basic structure of the entire to-be-trained inquiry diagnosis model is shown in FIG. 10.

The parameters may be specifically configured in such other way as may be sufficient to achieve the purpose of practical application, which is not limited herein.

In step S224, a to-be-trained palpation diagnosis model established based on a deep neural network algorithm is trained with the palpation diagnosis training data to obtain a trained palpation diagnosis model.

In the embodiment, the palpation diagnosis training data is collected by a digital pulse sensor HK-2000C designed by the Huake electronics research institute. Specifically, more preferable palpation diagnosis training data can be obtained by performing smoothing filtering preprocessing on the palpation diagnosis training data. It should be understood that by performing the above preprocessing, the inspection diagnosis training data is easier to be processed and analyzed in subsequent steps.

Specifically, in the embodiment, the palpation diagnosis training data inputted into the to-be-trained palpation diagnosis model is a collected pulse image, where the collected pulse image is a 3-channel image, that is, a RGB image. The to-be-trained palpation diagnosis model is established by using a deep neural network learning algorithm. It should be understood that data to be inputted into an input terminal of the to-be-trained palpation diagnosis model is the collected to-be-trained palpation diagnosis training data, and an output layer outputs a disease diagnosis result corresponding to the collected to-be-trained palpation diagnosis training data. The number of hidden layers and setting of specific parameters may be adjusted according to actual situations, which is not limited herein.

In step S23, disease data of the patient is diagnosed by the server with the trained model to obtain a diagnosis result of the disease data.

In the embodiment, disease information of the patient is classified, and deep neural network models corresponding to the to-be-trained inspection diagnosis training data, the to-be-trained auscultation-olfaction diagnosis training data, the to-be-trained inquiry diagnosis training data, and the to-be-trained palpation diagnosis training data are established respectively. It should be understood that with the method, data information about the disease of the patient can be more fully obtained, and the obtained information about disease of the patient is comprehensively analyzed, so that a diagnosis result of the disease of the patient can be obtained more accurately. Thus, the diagnosis result obtained with the method is more accurate than a diagnosis result by processing only tongue image information of the patient with a convolution neural network.

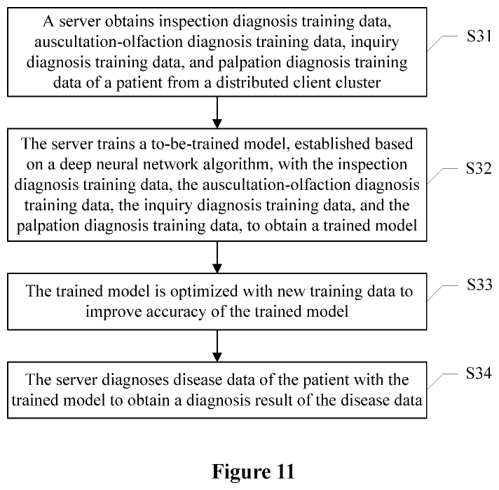

A specific intelligent traditional Chinese medicine diagnosis method is provided according to a third embodiment of the present disclosure. As shown in FIG. 11, compared with the foregoing embodiment, the technical solution is further explained and optimized in this embodiment, which includes the following steps S31 to S34.

In step S31, inspection diagnosis training data, auscultation-olfaction diagnosis training data, inquiry diagnosis training data, and palpation diagnosis training data of a patient are obtained by a sever from a distributed client cluster.

Reference may be made to the method according to the second embodiment for obtaining the inspection diagnosis training data, auscultation-olfaction diagnosis training data, inquiry diagnosis training data, and palpation diagnosis training data of the patient from a distributed client cluster in the embodiment, for which is thus not redundantly described herein.

In step S32, a to-be-trained model established based on a deep neural network algorithm is trained by the sever with the inspection diagnosis training data, the auscultation-olfaction diagnosis training data, the inquiry diagnosis training data, and the palpation diagnosis training data, to obtain a trained model. In the embodiment, this step specifically includes the following steps S321, S322, S323, S324, and S325.

In step S321, a to-be-trained inspection diagnosis model established based on a convolution neural network algorithm is trained with the inspection diagnosis training data to obtain a trained inspection diagnosis model.

In step S322, a to-be-trained auscultation-olfaction diagnosis model established based on a BP neural network algorithm is trained with the auscultation-olfaction diagnosis training data to obtain a trained auscultation-olfaction diagnosis model.

In step S323, a to-be-trained inquiry diagnosis model established based on a BP neural network algorithm is trained with the inquiry diagnosis training data to obtain a trained inquiry diagnosis model.

In step S324, a to-be-trained palpation diagnosis model established based on a deep neural network algorithm is trained with the palpation diagnosis training data to obtain a trained palpation diagnosis model.

It should be noted that reference may be made to the corresponding steps in the method according to the second embodiment of the present disclosure for the steps S321, S322, S323, and S324 in the embodiment, which is not redundantly described herein.

In step S325, a to-be-trained model established based on a probabilistic neural network algorithm is trained with data outputted from output terminals of the trained inspection diagnosis model, the trained auscultation-olfaction diagnosis model, the trained inquiry diagnosis model, and the trained palpation diagnosis model, to obtain a trained model.

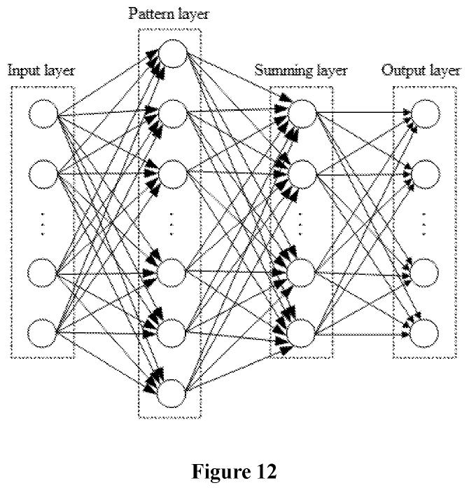

In the embodiment, the to-be-trained model is established based on a probabilistic neural network (Probabilistic Neural Network, PNN) algorithm. Disease diagnosis results obtained by the trained inspection diagnosis model, the trained auscultation-olfaction diagnosis model, the trained inquiry diagnosis model, and the trained palpation diagnosis model are used as input data of an input terminal of the to-be-trained model. A basic structure of a probabilistic neural network is shown in FIG. 12.

It should be understood that the diagnosis result of the disease of the patient can be more accurate by further providing a layer of deep neural network to optimize diagnosis results of the established models on the basis of the trained inspection diagnosis model, the trained auscultation-olfaction diagnosis model, the trained inquiry diagnosis model, and the trained palpation diagnosis model.

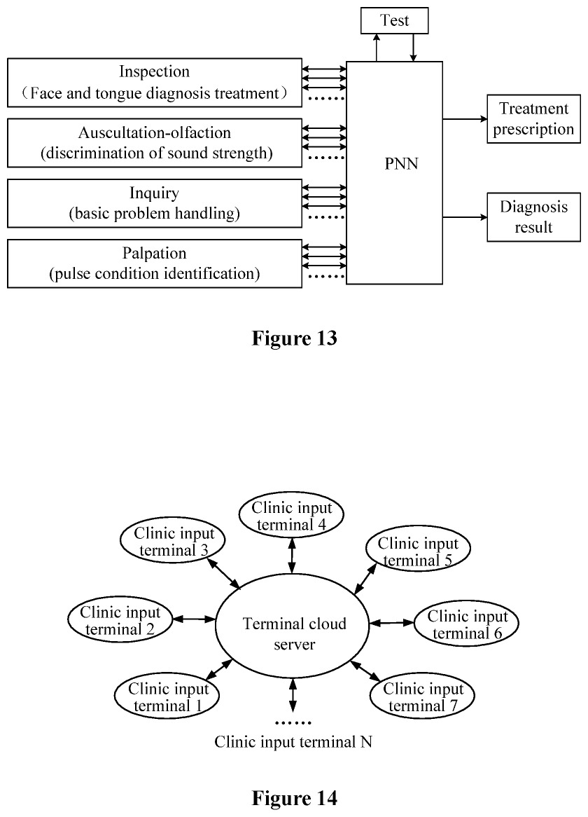

Specifically, in the embodiment, the number of neurons in an input layer of the to-be-trained model established based on the probabilistic neural network algorithm is equal to a sum of numbers of dimensions of vectors at output terminals of the trained inspection diagnosis model, the trained auscultation-olfaction diagnosis model, the trained inquiry diagnosis model, and the trained palpation diagnosis model. The entire model established based on the deep neural network algorithm is shown in FIG. 13.

It should be noted that in this step, the number and connection relationship of neurons and specific parameters in each layer in the established to-be-trained model are subject to adaption to purposes in practical application, where the parameters in the model are not limited.

In step S33, the trained model is optimized with new training data to improve accuracy of the trained model.

The new training data is disease data obtained after the diagnosis result of the patient is verified.

It should be understood that optimizing parameters of the trained model with the disease data obtained after the diagnosis result of the patient is verified can improve diagnosis accuracy of the model.

It should be noted that the training model may operate in two modes. One of the two modes is a training mode, where if the established model cannot independently diagnose diseases of the patient, the established model will be continuously trained with a large number of labeled data sets. The other is an operating and gradual optimizing mode of the model, where during normal use of the trained model, a corresponding diagnosis result will be given to the patient and the accuracy of the model will be continuously optimized with the disease data obtained after the diagnosis result of the patient is verified.

In step S34, disease data of the patient is diagnosed by the server with the trained model to obtain a diagnosis result of the disease data.

In the embodiment, corresponding models are established respectively for the inspection diagnosis training data, the auscultation-olfaction diagnosis training data, the inquiry diagnosis training data, and the palpation diagnosis training data first; on this basis, a to-be-trained model is established based on a probabilistic neural network; and data extracted from the output terminals of the trained inspection diagnosis model, the trained auscultation-olfaction diagnosis model, the trained inquiry diagnosis model and the trained palpation diagnosis model is used as input data of the input terminal of the to-be-trained model to train and optimize the disease data of the patient again.

It should be understood that with the method provided according to the embodiment, training accuracy of the model is significantly improved.

Reference is made to FIG. 14, which is a schematic diagram of a terminal cloud server based on the model. Specifically, in the embodiment, the terminal cloud server is connected to an input terminal of each clinic, where the inspection diagnosis training data, the auscultation-olfaction diagnosis training data, the inquiry diagnosis training data and the palpation diagnosis training data are respectively collected by an inspection diagnosis collecting terminal, an auscultation-olfaction diagnosis voice collecting terminal, an inquiry diagnosis information collecting terminal and a pulse collecting terminal of the cloud server. It should be noted that in the embodiment of the present disclosure, the disease data of the patient in the embodiments is all training data with data labels, where the data labels are set by the physician correspondingly when organizing the disease data of the patient.

It should be understood that by storing the inspection diagnosis training data, the auscultation-olfaction diagnosis training data, the inquiry diagnosis training data, and the palpation diagnosis training data in the cloud server, the established model can have the ability of training large-scale data. Moreover, in this way, the established model can diagnose multiple patients simultaneously, greatly improving practical performance of the model.

Furthermore, the cloud server may first detect through related settings whether there is a case library of the patient in the cloud server. If there is a case library of the patient in the cloud server, a normal disease data diagnosis process is performed. If there is no case library of the patient in the cloud server, a complete disease database will be automatically established for the patient in the system. In addition, with the system, a prescription of a treatment plan of the patient and precautions for the patient in daily life can be printed at a terminal of the system.

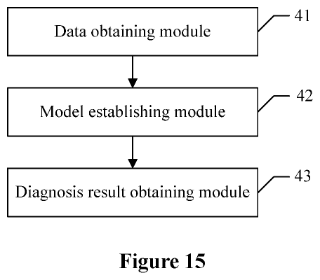

Accordingly, an intelligent traditional Chinese medicine diagnosis system is further provided according to an embodiment of the present disclosure, which is specifically configured on a cloud computing-based distributed client-server architecture. As shown in FIG. 15, the system includes:

a data obtaining module 41, configured for a server to obtain inspection diagnosis training data, auscultation-olfaction diagnosis training data, inquiry diagnosis training data, and palpation diagnosis training data of a patient from a distributed client cluster;

a model establishing module 42, configured for the server to train a to-be-trained model, established based on a deep neural network algorithm, with the inspection diagnosis training data, the auscultation-olfaction diagnosis training data, the inquiry diagnosis training data, and the palpation diagnosis training data, to obtain a trained model; and

a diagnosis result obtaining module 43, configured for the server to diagnose disease data of the patient with the trained model to obtain a diagnosis result of the disease data.

Specifically, the model establishing module 42 includes an inspection diagnosis establishing unit, an auscultation-olfaction diagnosis establishing unit, an inquiry diagnosis establishing unit, and a palpation diagnosis establishing unit.

The inspection diagnosis establishing unit is configured to train a to-be-trained inspection diagnosis model, established based on a convolution neural network algorithm, with the inspection diagnosis training data, to obtain a trained inspection diagnosis model.

The auscultation-olfaction diagnosis establishing unit is configured to train a to-be-trained auscultation-olfaction diagnosis model, established based on a BP neural network algorithm, with the auscultation-olfaction diagnosis training data, to obtain a trained auscultation-olfaction diagnosis model.

The inquiry diagnosis establishing unit is configured to train a to-be-trained inquiry diagnosis model, established based on a BP neural network algorithm, with the inquiry diagnosis training data, to obtain a trained inquiry diagnosis model.

The palpation establishing diagnosis unit is configured to train a to-be-trained palpation diagnosis model, established based on a deep neural network algorithm, with the palpation diagnosis training data, to obtain a trained diagnosis palpation model.

More specifically, the model establishing module 42 includes an inspection diagnosis establishing unit, an auscultation-olfaction diagnosis establishing unit, an inquiry diagnosis establishing unit, a palpation diagnosis establishing unit, and a model establishing unit.

The inspection diagnosis establishing unit is configured to train a to-be-trained inspection diagnosis model, established based on a convolution neural network algorithm, with the inspection diagnosis training data, to obtain a trained inspection diagnosis model.

The auscultation-olfaction diagnosis establishing unit is configured to train a to-be-trained auscultation-olfaction diagnosis model, established based on a BP neural network algorithm, with the auscultation-olfaction diagnosis training data, to obtain a trained auscultation-olfaction diagnosis model.

The inquiry diagnosis establishing unit is configured to train a to-be-trained inquiry diagnosis model, established based on a BP neural network algorithm, with the inquiry diagnosis training data, to obtain a trained inquiry diagnosis model.

The palpation diagnosis establishing unit is configured to train a to-be-trained palpation diagnosis model, established based on a deep neural network algorithm, with the palpation diagnosis training data, to obtain a trained palpation diagnosis model.

The model establishing unit is configured to train a to-be-trained model, established based on a probabilistic neural network algorithm, with data outputted from output terminals of the trained inspection diagnosis model, the trained auscultation-olfaction diagnosis model, the trained inquiry diagnosis model, and the trained palpation diagnosis model, to obtain a trained model.

Further, the intelligent traditional Chinese medicine diagnosis system according to an embodiment of the present disclosure further includes an inspection diagnosis data preprocessing module and an auscultation-olfaction diagnosis data preprocessing module.

The inspection diagnosis data preprocessing module is configured to perform denoising and/or smoothing processing on the inspection diagnosis training data.

The auscultation-olfaction diagnosis data preprocessing module is configured to perform filtering and/or framing processing on the auscultation-olfaction diagnosis training data.

Further, the intelligent traditional Chinese medicine diagnosis system according to an embodiment of the present disclosure further includes a model optimizing module.

The model optimizing module is configured to optimize, before the diagnosis result obtaining module 43 diagnoses the disease data of the patient with the trained model, the trained model with new training data to improve accuracy of the trained model.

The new training data is disease data obtained after the diagnosis result of the patient is verified.

Reference may be made to counterparts in the foregoing embodiments of the present disclosure for detailed operating of the modules and units described above, which is not redundantly described herein.

Accordingly, a traditional Chinese medicine medical system is further provided according to an embodiment of the present disclosure, including the intelligent traditional Chinese medicine diagnosis system described above. The traditional Chinese medicine medical system further includes:

an intelligent traditional Chinese medicine treatment system, configured to determine a corresponding treatment plan based on the diagnosis result obtained by the intelligent traditional Chinese medicine diagnosis system,

where the intelligent traditional Chinese medicine treatment system is a treatment system trained by using a deep neural network algorithm, and a corresponding training sample includes a history diagnosis result and a corresponding treatment plan.

It should be understood that the diagnosis result in the embodiment is substantively a classification result obtained by an information processing device such as a computer based on deep learning, and is different from a diagnosis conclusion obtained by a physician based on medical theories.

In the system, based on the diagnosis result of the patient, a treatment plan is determined for the patient by the treatment system trained by using a deep neural network algorithm. In order to obtain a better treatment plan, the system may be optimized with new training samples, which is not limited here.

Specifically, the treatment plan determined by the intelligent traditional Chinese medicine treatment system includes a prescription of Chinese traditional patent medicine and/or a physical therapy plan.

In the embodiment, the treatment plan determined by the intelligent traditional Chinese medicine treatment system includes, but is not limited to, a prescription of Chinese traditional patent medicine and/or a physical therapy plan. In this way, not only the workload of the physician can be reduced, but also a reference treatment plan can be provided for diagnosis results of patients, improving treatment experience for patients.

Specifically, the deep neural network algorithm used for training the intelligent traditional Chinese medicine treatment system includes a convolution neural network algorithm.

In the embodiment, the intelligent traditional Chinese medicine treatment system is obtained by virtue of advantages of simple structure, few training parameters, and strong adaptability of the convolutional neural network algorithm. In practical application, other deep neural network algorithms may also be used, which is not limited here.

In the embodiments of the present disclosure, the intelligent traditional Chinese medicine diagnosis system mainly determines the disease of the patient based on the disease information of the patient, and the intelligent traditional Chinese medicine treatment system can provide the patient with a corresponding disease diagnosis plan based on the disease determined by the above intelligent traditional Chinese medicine diagnosis system. In addition, the intelligent traditional Chinese medicine treatment system can flexibly change a proportional weight of a drug in a drug treatment plan for the patient based on different conditions of the patient. Further, with the system, a prescription of the treatment plan of the patient and precautions for the patient in daily life can be printed at a terminal of the system.

Finally, it should be further noted that in this context, relational terms such as first and second are used merely to distinguish one entity or operation from another entity or operation, and do not necessarily require or imply any such actual relationship or order between these entities or operations. Furthermore, the term “including”, “comprising” or any other variations thereof are intended to cover a non-exclusive inclusion, so that a process, method, item or apparatus including a series of elements includes not only those elements, but also other elements not explicitly listed, or elements inherent in such a process, method, item or apparatus. Without further limitation, an element preceded by the phrase “including a . . . ” does not exclude the existence of additional identical elements in the process, method, article or apparatus including the element.

The intelligent traditional Chinese medicine diagnosis method and system according to the present disclosure are described in detail above. The principles and implementations are clarified by using some specific embodiments herein. The above description of the embodiments is only intended to help understand the method of the present disclosure and the core idea thereof. In addition, changes can be made to the specific embodiments and the application scope by those skilled in the art based on the concept of the present disclosure. Therefore, the specification shall not be interpreted as limiting the present disclosure.

1. An intelligent traditional Chinese medicine diagnosis method, comprising:
obtaining, by a sever, inspection diagnosis training data, auscultation-olfaction diagnosis training data, inquiry diagnosis training data, and palpation diagnosis training data of a patient from a distributed client cluster;
training, by the sever, a to-be-trained model, established based on a deep neural network algorithm, with the inspection diagnosis training data, the auscultation-olfaction diagnosis training data, the inquiry diagnosis training data and the palpation diagnosis training data, to obtain a trained model; and
diagnosing, by the server, disease data of the patient with the trained model to obtain a diagnosis result of the disease data.

2. The method according to claim 1, wherein the method is implemented in a distributed client-server architecture or a cloud computing architecture.

3. The method according to claim 1, wherein the training a to-be-trained model, established based on a deep neural network algorithm, with the inspection diagnosis training data, the auscultation-olfaction diagnosis training data, the inquiry diagnosis training data and the palpation diagnosis training data, to obtain a trained model, comprises:
training a to-be-trained inspection diagnosis model, established based on a convolution neural network algorithm, with the inspection diagnosis training data, to obtain a trained inspection diagnosis model;
training a to-be-trained auscultation-olfaction diagnosis model, established based on a BP neural network algorithm, with the auscultation-olfaction diagnosis training data, to obtain a trained auscultation-olfaction diagnosis model;
training a to-be-trained inquiry diagnosis model, established based on a BP neural network algorithm, with the inquiry diagnosis training data, to obtain a trained inquiry diagnosis model; and
training a to-be-trained palpation diagnosis model, established based on a deep neural network algorithm, with the palpation diagnosis training data, to obtain a trained palpation diagnosis model.

4. The method according to claim 1, wherein the training a to-be-trained model, established based on a deep neural network algorithm, with the inspection diagnosis training data, the auscultation-olfaction diagnosis training data, the inquiry diagnosis training data and the palpation diagnosis training data, to obtain a trained model, comprises:
training a to-be-trained inspection diagnosis model, established based on a convolution neural network algorithm, with the inspection diagnosis training data, to obtain a trained inspection model;
training a to-be-trained auscultation-olfaction diagnosis model, established based on a BP neural network algorithm, with the auscultation-olfaction diagnosis training data, to obtain a trained auscultation-olfaction diagnosis model;
training a to-be-trained inquiry diagnosis model, established based on a BP neural network algorithm, with the inquiry diagnosis training data, to obtain a trained inquiry diagnosis model;
training a to-be-trained palpation diagnosis model, established based on a deep neural network algorithm, with the palpation diagnosis training data, to obtain a trained palpation diagnosis model; and
training a to-be-trained model, established based on a probabilistic neural network algorithm, with data outputted from output terminals of the trained inspection diagnosis model, the trained auscultation-olfaction diagnosis model, the trained inquiry diagnosis model, and the trained palpation diagnosis model, to obtain a trained model.

5. The method according to claim 1, further comprising:
performing denoising and/or smoothing processing on the inspection diagnosis training data.

6. The method according to claim 1, further comprising:
performing filtering and/or framing processing on the auscultation-olfaction diagnosis training data.

7. The method according to claim 1, wherein before the diagnosing disease data of the patient with the trained model to obtain a diagnosis result of the disease data, the method further comprises:
optimizing the trained model with new training data to improve accuracy of the trained model,
wherein the new training data is disease data obtained after the diagnosis result of the patient is verified.

8. An intelligent traditional Chinese medicine diagnosis system, comprising:
a data obtaining module, configured for a server to obtain inspection diagnosis training data, auscultation-olfaction diagnosis training data, inquiry diagnosis training data, and palpation diagnosis training data of a patient from a distributed client cluster;
a model establishing module, configured for the server to train a to-be-trained model, established based on a deep neural network algorithm, with the inspection diagnosis training data, the auscultation-olfaction diagnosis training data, the inquiry diagnosis training data, and the palpation diagnosis training data, to obtain a trained model; and
a diagnosis result obtaining module, configured for the server to diagnose disease data of the patient with the trained model to obtain a diagnosis result of the disease data.

9. A traditional Chinese medicine medical system, comprising:
the intelligent traditional Chinese medicine diagnosis system according to claim 8, and
an intelligent traditional Chinese medicine treatment system, configured to determine a corresponding treatment plan based on the diagnosis result obtained by the intelligent traditional Chinese medicine diagnosis system,
wherein the intelligent traditional Chinese medicine treatment system is a treatment system trained by using a deep neural network algorithm, and a corresponding training sample comprises a history diagnosis result and a corresponding treatment plan.

10. The traditional Chinese medicine medical system according to claim 9, wherein
the treatment plan determined by the intelligent traditional Chinese medicine treatment system comprises a prescription of Chinese traditional patent medicine and/or a physical therapy plan.

11. The traditional Chinese medicine medical system according to claim 9, wherein
the deep neural network algorithm used for training the intelligent traditional Chinese medicine treatment system comprises a convolution neural network algorithm.

12. The method according to claim 2, wherein before the diagnosing disease data of the patient with the trained model to obtain a diagnosis result of the disease data, the method further comprises:
optimizing the trained model with new training data to improve accuracy of the trained model,
wherein the new training data is disease data obtained after the diagnosis result of the patient is verified.

13. The method according to claim 3, wherein before the diagnosing disease data of the patient with the trained model to obtain a diagnosis result of the disease data, the method further comprises:
optimizing the trained model with new training data to improve accuracy of the trained model,
wherein the new training data is disease data obtained after the diagnosis result of the patient is verified.

14. The method according to claim 4, wherein before the diagnosing disease data of the patient with the trained model to obtain a diagnosis result of the disease data, the method further comprises:
optimizing the trained model with new training data to improve accuracy of the trained model,
wherein the new training data is disease data obtained after the diagnosis result of the patient is verified.

15. The intelligent traditional Chinese medicine diagnosis system according to claim 8, wherein the model establishing module comprises an inspection diagnosis establishing unit, an auscultation-olfaction diagnosis establishing unit, an inquiry diagnosis establishing unit, and a palpation diagnosis establishing unit, wherein:
the inspection diagnosis establishing unit is configured to train a to-be-trained inspection diagnosis model, established based on a convolution neural network algorithm, with the inspection diagnosis training data, to obtain a trained inspection diagnosis model;
the auscultation-olfaction diagnosis establishing unit is configured to train a to-be-trained auscultation-olfaction diagnosis model, established based on a BP neural network algorithm, with the auscultation-olfaction diagnosis training data, to obtain a trained auscultation-olfaction diagnosis model;
the inquiry diagnosis establishing unit is configured to train a to-be-trained inquiry diagnosis model, established based on a BP neural network algorithm, with the inquiry diagnosis training data, to obtain a trained inquiry diagnosis model; and
the palpation establishing diagnosis unit is configured to train a to-be-trained palpation diagnosis model, established based on a deep neural network algorithm, with the palpation diagnosis training data, to obtain a trained diagnosis palpation model.

16. The intelligent traditional Chinese medicine diagnosis system according to claim 8, wherein the model establishing module comprises an inspection diagnosis establishing unit, an auscultation-olfaction diagnosis establishing unit, an inquiry diagnosis establishing unit, a palpation diagnosis establishing unit, and a model establishing unit, wherein:
the inspection diagnosis establishing unit is configured to train a to-be-trained inspection diagnosis model, established based on a convolution neural network algorithm, with the inspection diagnosis training data, to obtain a trained inspection diagnosis model;
the auscultation-olfaction diagnosis establishing unit is configured to train a to-be-trained auscultation-olfaction diagnosis model, established based on a BP neural network algorithm, with the auscultation-olfaction diagnosis training data, to obtain a trained auscultation-olfaction diagnosis model;
the inquiry diagnosis establishing unit is configured to train a to-be-trained inquiry diagnosis model, established based on a BP neural network algorithm, with the inquiry diagnosis training data, to obtain a trained inquiry diagnosis model;
the palpation establishing diagnosis unit is configured to train a to-be-trained palpation diagnosis model, established based on a deep neural network algorithm, with the palpation diagnosis training data, to obtain a trained diagnosis palpation model; and
the model establishing unit is configured to train a to-be-trained model, established based on a probabilistic neural network algorithm, with data outputted from output terminals of the trained inspection diagnosis model, the trained auscultation-olfaction diagnosis model, the trained inquiry diagnosis model, and the trained palpation diagnosis model, to obtain a trained model.

17. The intelligent traditional Chinese medicine diagnosis system according to claim 8, further comprising an inspection diagnosis data preprocessing module and an auscultation-olfaction diagnosis data preprocessing module, wherein:
the inspection diagnosis data preprocessing module is configured to perform denoising and/or smoothing processing on the inspection diagnosis training data; and
the auscultation-olfaction diagnosis data preprocessing module is configured to perform filtering and/or framing processing on the auscultation-olfaction diagnosis training data.

18. The intelligent traditional Chinese medicine diagnosis system according to claim 8, further comprising a model optimizing module,
wherein the model optimizing module is configured to optimize, before the diagnosis result obtaining module diagnoses the disease data of the patient with the trained model, the trained model with new training data to improve accuracy of the trained model,
wherein the new training data is disease data obtained after the diagnosis result of the patient is verified.

19. The intelligent traditional Chinese medicine diagnosis system according to claim 15, further comprising a model optimizing module,
wherein the model optimizing module is configured to optimize, before the diagnosis result obtaining module diagnoses the disease data of the patient with the trained model, the trained model with new training data to improve accuracy of the trained model,
wherein the new training data is disease data obtained after the diagnosis result of the patient is verified.

20. The intelligent traditional Chinese medicine diagnosis system according to claim 16, further comprising a model optimizing module,
wherein the model optimizing module is configured to optimize, before the diagnosis result obtaining module diagnoses the disease data of the patient with the trained model, the trained model with new training data to improve accuracy of the trained model,
wherein the new training data is disease data obtained after the diagnosis result of the patient is verified.
